(19) United States
(12) Patent Application Publication
(10) Pub. No.: US 2007/0235348 A1
(43) Pub. Date: Oct. 11, 2007

(54) Conducting polymer nanosensor

(76) Inventors: Larry A. Nagahara, Phoenix, AZ (US); Islamshah S. Amlani, Chandler, AZ (US); Alvaro Diaz Aguilar, Tempe, AZ (US); Erica S. Forzani, Tempe, AZ (US); Xiulan Li, Tempe, AZ (US); Nongjian Tao, Scottsdale, AZ (US)

Correspondence Address: INGRASSIA FISHER & LORENZ, P.C., 7150 E. CAMELBACK, STE. 325, SCOTTSDALE, AZ, 85251, US

(21) Appl. No.: 11/399,834

(22) Filed: Apr. 7, 2006

(51) Int. Cl. G01F 1/64; G01N 33/487

(52) U.S. Cl. 205/792; 204/403.01

(57) ABSTRACT

A method is provided for forming a thin film conducting polymer (24) for sensor applications. The method comprises forming at least one pair of electrodes (14, 16) on a substrate (12), the pair of electrodes (14, 16) having an insulating layer (18) positioned therebetween, the insulating layer (18) having a surface (20) opposed to the substrate (12), increasing OH⁻ groups on the surface (20), binding silane molecules (22) to the surface (20), and forming the conducting polymer material (24) on the silane molecules (22) between and in electrical contact with the electrodes (14, 16).

### FIELD OF THE INVENTION

The present invention generally relates to conducting polymer formation and more particularly to a method of forming a thin film conducting polymer for sensor applications.

### BACKGROUND OF THE INVENTION

Conducting polymers includes several types of polymeric materials with electronic and/or ionic conductivity. A number of different monomers can be used to produce different conducting polymers. Nobel laureates Heeger, MacDiarmid and Shiragawa demonstrated that the molecular arrangement in conducting polymers must contain alternating single and double bonds (i.e., conjugated-electron systems along their backbone) in order to allow the formation of delocalized electronic states which leads to the formation of an energy band gap. Most conducting polymers, such as polyaniline (PANI), and polypyrrole (PPY) are p-type semiconductors. The primary dopants (anions) can be introduced during a chemical or electrochemical polymerization of the monomer. Extrinsic conducting polymers (i.e., composites) can also be fabricated by combining conducting polymers and/or with conductive fillers (e.g., conductive powders or metallic nanoparticles) with more insulating polymers. Intrinsic and extrinsic conducting polymers can be fabricated from a variety of techniques, for example, electropolymerization, polymer grafting, compression molding, and spray coating.

Conducting polymers are promising materials for a large variety of chemical and biological sensor applications, as the polymer properties are altered in response to various analytes. Conducting polymers can be used as the selective layer in sensors or as the transducer itself. Conducting polymer sensors based on measuring shifts in work function, changes in optical absorption spectra, and electrical conductance changes are known.

The most common sensing platform of conducting polymers is based on changes in conductance, such as chemiresistors or chem-field effect transistors (ChemFETs). In its simplest form, a chemiresistor comprises a conducting polymer layer deposited on an insulating surface with a pair of metal electrodes forming contacts to the conducting polymer. When a constant potential is applied, the resulting current flowing between the electrodes becomes the response signal. As the conducting polymer interacts with analytes, it can act either as an electron donor or an electron acceptor. If a p-type conducting polymer donates electrons to the analyte its hole conductivity increases. Conversely, when the same conducting polymer acts as an electron acceptor its conductivity decreases.

Doping offers a powerful transduction mechanism since the polymer's conductivity can change by several orders of magnitude with even a small amount of analyte (charge injection). Hence, chemical selectivity coupled to this doping phenomenon is attractive approach for conducting polymer sensors. The charged nature of the carriers also lends itself to interactions with the surrounding medium, thereby affecting the conductivity. In this scheme, the analyte acts as chemical transducers or ‘secondary’ dopants in the conducting polymer system. The interactions of analyte with the conducting polymer, via electrostatic, hydrogen bonding, van der Waals forces, or covalent interaction, will modulate the electronic and/or optical properties of the conducting polymer. Other transduction mechanism in conducting polymers includes swelling of the polymer upon sorption of the analyte which can also affect the conductive pathways or percolation network in the conducting polymer layer.

In general, the measured change in the conducting polymer properties results from changes in bulk properties. The distinct disadvantages of the response originating in the bulk are the relatively long time constant (tens of seconds to minutes) and hysteresis in recycling the conducting polymer. These effects are caused by slow penetration of analytes into the conducting polymer.

On the other hand, reducing the sensor's dimensions (x, y, and z) can overcome many of these disadvantages. Small lattice distortions occurring locally as well as charge inequalities induced in the conducting polymer backbone generate a larger percentage change in the signal at the nanoscale and hence increase sensitivity of the sensor. The trend is toward smaller sensors with faster response and higher sensitivity. Scaling both the thickness and width of the conducting polymer provides for better conduction, faster response, higher sensitivity, multianalyte detection, and smaller form factor. However, as the dimensions of the conducting polymer shrinks, uniformity becomes more critical to response time and sensitivity.

The affinity and specificity to a particular chemical or biological analyte (and hence the conducting polymer sensor's selectivity and sensitivity) can be enhanced by incorporating peptides and/or aptamers into the conducting polymer layer. Peptides are short chains of amino acids while aptamers are short chains of single stranded DNA or RNA molecules. Peptides/aptamers can bind with analyte molecules depending on the sequence, length and/or resulting three-dimensional shape. The number of different oligopeptides/aptamers is virtually unlimited by choosing different amino acid/nucleotide sequences, which allows one to tune the specificity of the sensor via a combinatorial chemistry approach.

Accordingly, it is desirable to provide a method of forming a thin film conducting polymer for sensor applications. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A method is provided for forming a thin film conducting polymer for sensor applications. The method comprises forming at least one pair of electrodes on a substrate, the pair of electrodes having an insulating layer positioned therebetween, the insulating layer having a surface opposed to the substrate, increasing OH⁻ groups on the surface, binding silane molecules to the surface, and forming the conducting polymer material on the silane molecules between and in electrical contact with the electrodes.

### BRIEF DESCRIPTION OF THE DRAWINGS

The present invention will hereinafter be described in conjunction with the following drawing figures, wherein like numerals denote like elements, and

FIG. 1 is a partial top view of a structure fabricated using the method of the exemplary embodiment;

FIG. 2 is a partial side view of the structure taken along line 2-2 of FIG. 1;

FIG. 3 is a flow chart of the method of the exemplary embodiment;

FIG. 4 is a schematic representation showing the advantage of using the exemplary embodiment; and

FIG. 5 is a graph showing the advantage of using the exemplary embodiment.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

When an analyte attaches itself to or interacts with a conducting polymer material, a characteristic of the material changes, such as the change in a current flowing in the conducting polymer material that is measurable. Various sensing mechanisms have been proposed for conducting polymer as sensors. One such sensing mechanism stems from changes in charge density on the surface of the conducting polymer material, thereby affecting the carrier concentration inside the conducting polymer material. One or more conducting polymer material may also be fabricated as an interdigitated device. Additionally, the conducting polymer material may be coated with a substance (functionalized with molecule specific coating) for determining specific environmental agents. And while a change in current is the preferred embodiment for the measurable material characteristic, other embodiments would include, for example, magnetic, optical, frequency, and mechanical for measurable material characteristics.

By measuring this change in the current, it is known that a determination may be made as to the number of molecules that have attached to the conducting polymer material, and therefore, a correlation to the concentration of the molecules in the environment around the conducting polymer material. Known systems place at least one electrode couple the conducting polymer material to measure this change in the material characteristic.

While scaling the dimensional length scales in conducting polymer sensor platform toward nanoscale affords certain advantages over macroscopic conducting polymer sensors, such as improved conduction, faster response, higher sensitivity, multianalyte detection, and smaller form factor to name a few, scaling the sensor's dimensions creates numerous challenges. For example, uniformity of the conducting polymer film becomes critical as variation in film thickness plays a much more significant role in response time, sensitivity, etc. at the nanoscale as compared to a macroscopic conducting polymer film.

As the separation distance between adjacent electrodes is reduced, the interplay between directing the monomer toward the insulating surface and the affect of increasing electric field strength between the electrodes becomes more apparent. Increasing the affinity for the monomer to be on or adjacent to the insulating surface improves the properties of a nanoscale conducting polymer film for sensor applications.

Although improvements in sensor performance due to scaling have been made, these methods have been adequate for demonstrating the characteristics of individual devices, with little regards to the compatibility for manufacturing (e.g., ease, reproducibility and volume). Disclosed herein are methods of forming a thin conducting polymer for sensor applications that is facile, reproducible, highly sensitive and selective and more importantly compatible toward nanoscaling. One such example described below is the introduction of a novelty polymer layer between the substrate surface and conducting polymer layer, so as to enhance uniformity in conducting polymer formation during electropolymerization. Although the exemplary embodiment describes a conductive polymer, any conductive organic material may be used in the invention. Examples of conductive polymers include polyaniline (PANI), polypyrrole (PPY), polythiophene, polyphenylene, polyacetylene, and derivatives thereof.

As subsequently described in more detail, the formation of the conducting polymer nanosensor comprises achieving uniform conducting polymer film formation using selective functionalization of the underlying substrate surface. The conducting polymer is formed by electropolymerization of a mixture of aniline and peptide modified-aniline in presence of poly (styrenesulfonic acid) to bridge two electrodes. The two electrodes are positioned on the substrate using standard lithographic techniques, with a silicon dioxide layer positioned between the electrodes on the substrate surface. The structure is boiled in deionized water to increase the OH⁻ groups at the surface of the silicon dioxide and then immersed in a silane solution in acetone which is bubbled continuously in nitrogen. The silane molecule binds to the native silicon oxide on the substrate. The structure is rinsed thoroughly in deionized water, dried with nitrogen, and then baked. The structure is immersed in poly acrylic acid solution. This process allows for conducting polymer formation on a nano-scale of 1 μm, or even to a dimension of single molecule.

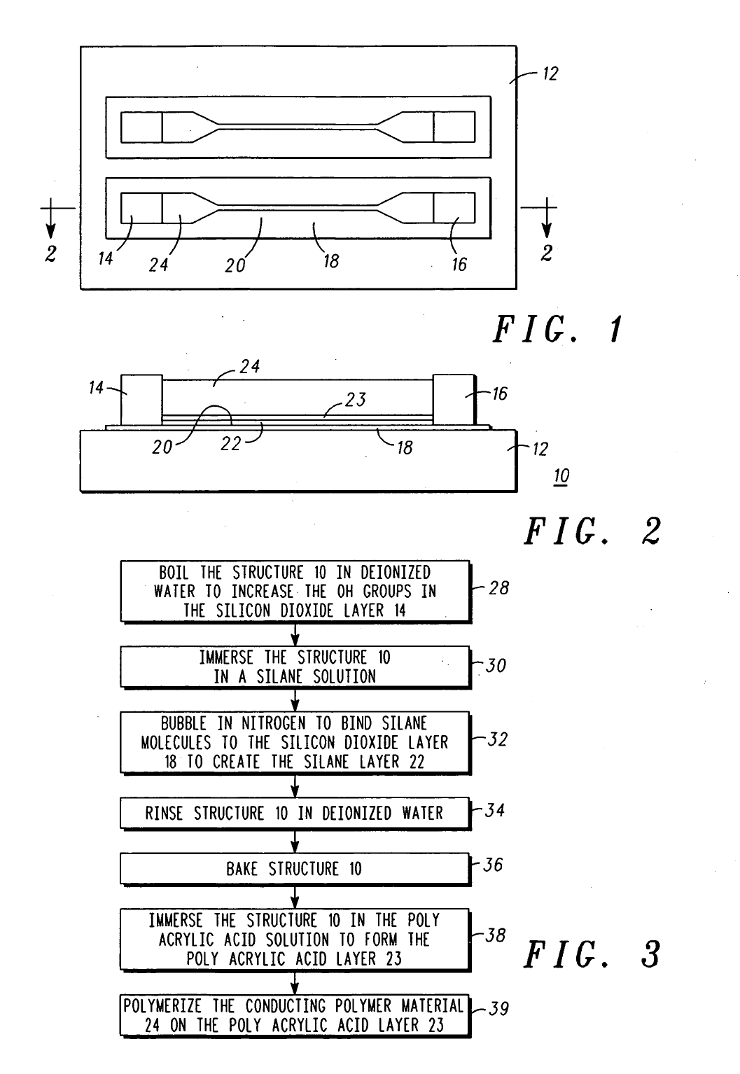

Referring to FIGS. 1 and 2, the structure 10 of the conducting polymer nanosensor includes a substrate 12 preferably comprising silicon; however, alternate materials, for example, quartz, sapphire, plastic, ceramic, metal, other semiconductor materials, or a flexible material are anticipated by this disclosure. Substrate 12 may include control electronics or other circuitry. An insulating layer 18, such as silicon dioxide, silicon nitride, or the like is formed, typically by deposition, on the substrate 12, but may also blanket the substrate 12. First and second electrodes 14, 16 comprise a conducting material, for example, a metal of gold, and preferably are separated by less than a 60 nanometer gap. The electrodes 14, 16 preferably are fabricated on the insulating layer 18 using electron beam lithography or gold electroplating on 1 μm gap electrodes to form the 60 nanometer gap. It should be understood that while two structures 10 are illustrated in the exemplary embodiment described herein, many hundreds or thousands may exist in arbitrary orientation on a single substrate.

The structure 10 is boiled 28 (see the flow chart in FIG. 3) in deionized water for about 30 minutes to increase the OH⁻ groups at the surface 20 of the insulating layer 18, and then immersed 30 in a silane solution, e.g., 250 micro liters of 3-aminopropyltrietoxysilane in 12 milliliters of acetone (2% silane solution) which is bubbled 32 continuously in nitrogen for several hours. The silane molecule binds to the silicon oxide layer 18 as a silane layer 22. The structure 10 is then rinsed 34 thoroughly in deionized water, dried with nitrogen, and then baked 36 at 80° C. for about 30 minutes. The structure 10 is immersed 38 in a poly acrylic acid solution forming a poly acrylic acid layer 23 (1 milliliter of deionized water and 250 microliters of poly acrylic acid for about 2 hours) for increasing the charge density. The conducting polymer material 24 is then polymerized 39 on the poly acrylic acid layer 23 between electrodes 14, 16. At an earlier point in the process, an insulating material (not shown) may be patterned on the electrodes 14, 16 to define a portion of the electrodes 14, 16 from which the conducting polymer material 24 is formed. Furthermore, the conducting polymer material 24 may be formed from both electrodes 14, 16, thereby meeting and making contact in between, e.g., the middle, or the conducting polymer material 24 may be formed from one of the electrodes 14, 16 to reach the other of the electrodes 14, 16.

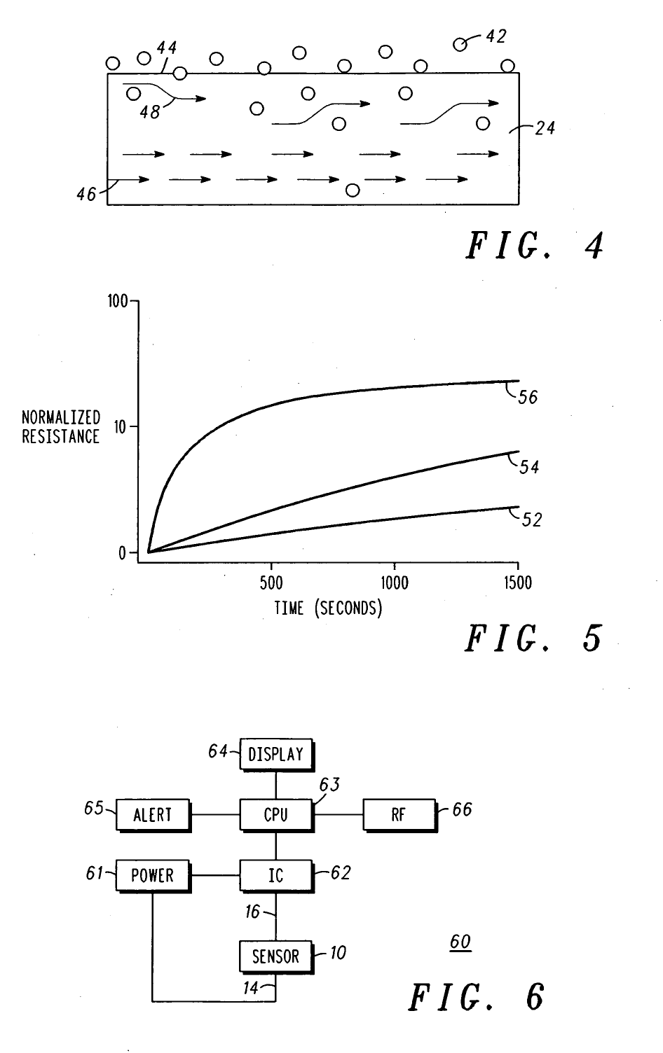

FIG. 4 is a representation of the conductive polymer material 24 having species 42 being absorbed within. It may be seen as the species 42 impacts the surface 44 of the conductive polymer material 24, more of the species 42 are positioned near the surface 44 than away from the surface 44. The current 46 flows smoothly in the conducting polymer material 24 where there is no or few species 42; however, where the species 42 is more numerous near the surface 44, the current 48 is impeded by the species 42. Therefore, the thinner the conducting polymer material 24 is, the species 42 will occupy more of the conducting polymer material 24, increasing the effect on the total current between the electrodes 14, 16.

FIG. 5 is a graph illustrates the sensitivity, or response time, of a conductive polymer material 24 being subjected to a species 26. Lines 52, 54, and 56 represent a conductive polymer material having a thickness of 1.0 micrometer, 0.7 micrometer, and 0.3 micrometer, respectively. It is seen that the smaller the thickness, the resistance increases in much less time, and at a greater amplitude, than the larger thicknesses.

Referring to FIG. 6, an exemplary system 60 includes the device 10, for example, having its electrodes 14 and 16 coupled to a power source 61, e.g., a battery. A circuit 62 determines the current between the electrodes and supplies the information to a processor 63. The information may be transferred from the processor 63 to a display 64, an alert device 65, and/or an RF transmitter 66.

Detection of metal ion species such as Cu²⁺ and Ni²⁺ can be achieved by using peptide modified-aniline (Gly-Gly-His (GGH-aniline) and (His)₆ (H6-aniline)), respectively. The gap between each pair of electrodes is bridged with polyaniline or peptide-modified polyaniline by cycling the electrode potential in 0.1 M aniline+supporting electrolyte solution (0.5 M NaHSO₄+10 mg/mL PSS+1.9 M H₂SO₄). The aniline solution contained either 100% regular aniline for the reference nanojunction, 75% GGH-aniline+15% aniline for GGH-modified nanojunction (for Cu²⁺) or 90% H6-aniline+10% aniline for (His) 6-modified nanojunction (for Ni²⁺).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims.

1. A method of forming a conducting polymer material between pairs of electrodes of a plurality of electrodes on a substrate, the pairs of electrodes having an insulating layer positioned therebetween on the substrate, the insulating layer having a surface opposed to the substrate, comprising:
binding molecules to the surface to obtain a desired affinity; and
forming the conducting organic material on the surface and coupled between the electrodes.

2. The method of claim 1 wherein the insulating layer comprises silicon dioxide and the binding step comprises increasing OH⁻ groups on the surface.

3. The method of claim 1 wherein the binding step comprises immersing in a silane solution subsequent to the increasing step.

4. The method of claim 3 further comprising bubbling in nitrogen prior to the forming step.

5. The method of claim 3 further comprising immersing in polyacrylic acid solution prior to the forming step.

6. The method of claim 3 wherein the immersing step comprises immersing in 3-aminopropyltrietoxysilane and acetone.

7. The method of claim 6 wherein the forming step comprises forming a conducting polymer material.

8. The method of claim 1 wherein the forming step comprises forming a conducting polymer material

9. The device of claim 1 further comprising determining an electrical change in the conducting organic layer when the conducting organic layer is exposed to analytes.

10. The device of claim 9 wherein the analytes comprise one of chemical and biological species.

11. The device of claim 1 further comprising treating the conducting organic layer with at least one of peptides and aptamers.

12. The device of claim 11 wherein the peptides and aptamers comprise selected sequences and lengths.

13. The device of claim 12 further comprising tuning the sequences and lengths with combinatorial chemistry approaches to optimize the selectivity, sensitivity and response time of the device.

14. A method of fabricating a structure, comprising:
forming at least a pair of electrodes on a substrate of the structure, wherein an insulating layer is positioned on the substrate between the at least a pair of electrodes, the insulating layer having a surface opposed to the substrate;
increasing OH⁻ groups on the surface;
immersing the structure in a silane solution to form a silane layer on the surface;
bubbling the structure in nitrogen;
rinsing the structure in deionized water;
drying the structure;
baking the structure;
immersing the structure in polyacrylic acid solution to form a polyacrylic acid layer on the silane layer; and
forming a conductive organic layer on the polyacrylic acid layer and coupled between the pair of electrodes.

15. The method of claim 14 wherein the insulating layer comprises an oxide layer.

16. The method of claim 14 wherein the forming step comprises forming a conductive polymer.

17. A method of forming a conducting polymer material between pairs of electrodes of a plurality of electrodes on a substrate, the pairs of electrodes having an oxide layer positioned therebetween and on the substrate and having a surface opposed to the substrate, comprising:
boiling in deionized water to increase OH⁻ groups on the surface;
immersing in a silane solution;
bubbling in nitrogen to bind silane molecules to the surface;
rinsing in deionized water;
drying;
baking; and
immersing in polyacrylic acid solution to form a polyacrylic acid layer on the silane molecules; and
forming a conductive organic layer on the polyacrylic acid layer and coupled between the pair of electrodes.

18. A device comprising:
a substrate; and
one or more pairs of electrodes positioned on and electrically isolated from the substrate, each pair of electrodes comprising;
an insulating layer formed on the substrate between the pair of electrodes;
a silane layer formed on the insulating layer;
a poly acrylic acid layer formed on the silane layer; and
a conducting organic layer formed on the poly acrylic acid layer and coupled between the pair of electrodes.

19. The device of claim 18 wherein the insulating layer comprises an oxide.

20. The device of claim 18 wherein the conducting organic layer comprises a conducting polymer material.

21. The device of claim 18 further comprising circuitry for determining an electrical change in the conducting organic layer when the conducting organic layer is exposed to analytes.

22. The device of claim 18 wherein the analytes comprise one of chemical and biological species.

23. The device of claim 18 wherein the conducting organic layer includes at least one of peptides and aptamers.

24. The device of claim 23 wherein the peptides and aptamers comprise selected sequences and lengths.
